(19) United States
(12) Patent Application Publication
(10) Pub. No.: US 2026/0071246 A1
(43) Pub. Date: Mar. 12, 2026

(54) HIGHLY-ACTIVE RHIZOMA DIOSCOREAE POLYSACCHARIDE AND PREPARATION METHOD THEREOF

(71) Applicant: SERICULTURAL & AGRI-FOOD RESEARCH INSTITUTE GUANGDONG ACADEMY OF AGRICULTURAL SCIENCES, Guangzhou (CN)

(72) Inventors: Fei HUANG, Guangzhou (CN); Ruifen ZHANG, Guangzhou (CN); Mingwei ZHANG, Guangzhou (CN); Xuchao JIA, Guangzhou (CN); Mei DENG, Guangzhou (CN); Qin MA, Guangzhou (CN); Lihong DONG, Guangzhou (CN); Dong ZHAO, Guangzhou (CN); Shan LIANG, Guangzhou (CN); Yanxia CHEN, Guangzhou (CN); Yuan ZHANG, Guangzhou (CN)

(21) Appl. No.: 19/389,282

(22) Filed: Nov. 14, 2025

Related U.S. Application Data
(63) Continuation of application No. PCT/CN2024/080944, filed on Mar. 11, 2024.

(30) Foreign Application Priority Data
Jan. 23, 2024 (CN) 202410091396.9

(51) Int. Cl. C12P 19/04

(52) U.S. Cl. CPC C12P 19/04; C12P 2203/00

(57) ABSTRACT

A highly-active Rhizoma dioscoreae polysaccharide and a preparation method thereof is described in relation to nutritional food production. The preparation method includes: subjecting a Rhizoma dioscoreae slurry to an ultra-high pressure treatment to produce an ultra-high pressure-treated Rhizoma dioscoreae slurry; inoculating Lactiplantibacillus plantarum subsp. plantarum into the ultra-high pressure-treated Rhizoma dioscoreae slurry, and conducting fermentation to produce a Rhizoma dioscoreae fermentation broth; and subjecting the Rhizoma dioscoreae fermentation broth to hot water extraction and then centrifugation, and collecting, concentrating, and drying a resulting supernatant to produce the Rhizoma dioscoreae polysaccharide.

### CROSS REFERENCE TO RELATED APPLICATIONS

This application is a bypass continuation of International Patent Application No. PCT/CN2024/080944, filed on Mar. 11, 2024, which claims priority of the Chinese Patent Application No. 202410091396.9, filed on Jan. 23, 2024, both of which are incorporated by references in their entities.

### TECHNICAL FIELD

The present disclosure belongs to the technical field of nutritional food production, and specifically relates to a highly-active Rhizoma dioscoreae polysaccharide and a preparation method thereof.

### BACKGROUND

Rhizoma dioscoreae, also known as Chinese yam or Nagaimo, is rich in nutrients and possesses various physiological health benefits. Thus, Rhizoma dioscoreae is an excellent medicinal and dietary product. According to traditional Chinese medicine, Rhizoma dioscoreae is believed to have functions such as strengthening the spleen and arresting diarrhea. Studies on modern medicine have shown that polysaccharides are the major active functional components in Rhizoma dioscoreae, and demonstrate effects such as immune regulation, diabetes improvement, and antibacterial effect. As a result, the research related to Rhizoma dioscoreae polysaccharides has attracted widespread attention. Rhizoma dioscoreae polysaccharides may alleviate the pathological damage to colons in mice with dextran sulfate sodium (DSS)-induced colitis, inhibit the activation of the inflammatory signaling pathway NF-κB in colons, and restore the mRNA expression of the tight junction protein ZO-1, thereby relieving colitis. Additionally, Rhizoma dioscoreae polysaccharides may increase the insulin secretion and improve the impairment of functional pancreatic β-cells in diabetic rats, thereby playing a hypoglycemic role. Rhizoma dioscoreae polysaccharides, as natural active functional factors, hold promising prospects for development and utilization. However, the high starch content in Rhizoma dioscoreae significantly hinders the dissolution of Rhizoma dioscoreae polysaccharides. Since Rhizoma dioscoreae polysaccharides are present in cell walls, components such as cellulose and hemicellulose in Rhizoma dioscoreae also impede the dissolution of Rhizoma dioscoreae polysaccharides. Currently, extraction techniques for Rhizoma dioscoreae polysaccharides include hot water extraction, ultrasound-assisted extraction, enzyme-assisted extraction, etc. However, the hot water extraction has relatively-low efficiency. In addition, the remaining methods are only suitable for small-scale polysaccharide preparation in the laboratory, and require high energy consumption, making these methods unsuitable for large-scale production. The disadvantages such as low extraction yield and high energy consumption of these Rhizoma dioscoreae polysaccharide extraction techniques restrict the development and utilization of Rhizoma dioscoreae. Therefore, there is an urgent need to develop a new Rhizoma dioscoreae polysaccharide extraction technique to address this issue.

### SUMMARY

The present disclosure provides a highly-active Rhizoma dioscoreae polysaccharide and a preparation method thereof. A Rhizoma dioscoreae polysaccharide prepared by the preparation method of the present disclosure exhibits a high yield and a significant inhibitory activity against lipase and amylase. Moreover, the preparation method offers high extraction efficiency and low energy consumption, and is environmentally friendly.

To solve the above technical problem, the present disclosure provides the following technical solutions:

The present disclosure provides a method for preparing a highly-active Rhizoma dioscoreae polysaccharide, including the following steps: subjecting a Rhizoma dioscoreae slurry to ultra-high pressure treatment to produce an ultra-high pressure-treated Rhizoma dioscoreae slurry; inoculating Lactiplantibacillus plantarum subsp. plantarum into the ultra-high pressure-treated Rhizoma dioscoreae slurry, and conducting fermentation to produce a Rhizoma dioscoreae fermentation broth; and subjecting the Rhizoma dioscoreae fermentation broth to hot water extraction and then centrifugation to obtain a supernatant, concentrating the supernatant to obtain a concentrated solution, and drying the concentrated solution to produce the Rhizoma dioscoreae polysaccharide.

Preferably, the method further comprises mixing fresh Rhizoma dioscoreae and water at a mass ratio of 1:0.5-3 to obtain a mixture, and pulping the mixture to produce an preliminary Rhizoma dioscoreae slurry.

Preferably, the method further comprises mixing the preliminary Rhizoma dioscoreae slurry and water to produce the Rhizoma dioscoreae slurry before the ultra-high pressure treatment; and in the Rhizoma dioscoreae slurry, a mass-to-volume ratio of the fresh Rhizoma dioscoreae to total water is 1:4 to 1:8.

Preferably, the ultra-high pressure treatment is conducted under a pressure of 200 MPa to 600 MPa with a pressure-holding time of 5 min to 11 min.

Preferably, the Lactiplantibacillus plantarum subsp. plantarum needs to be cultured in an MRS broth medium to produce a bacterial suspension for use.

Preferably, concentration of the Lactiplantibacillus plantarum subsp. plantarum is 0.5×10⁹ cfu/mL to 1.5×10⁹ cfu/mL.

Preferably, a inoculation percentage of a mass of fresh Rhizoma dioscoreae to a volume of a suspension of the Lactiplantibacillus plantarum subsp. plantarum is 2% to 6%.

Preferably, the fermentation is conducted at 35° C. to 40° C.

Preferably, the fermentation is conducted for 1 d to 4 d.

Preferably, before the Lactiplantibacillus plantarum subsp. plantarum is inoculated for the fermentation, the ultra-high pressure-treated Rhizoma dioscoreae slurry needs to be pasteurized at 70° C. to 90° C. for 20 min to 40 min.

Preferably, water is added at a final amount of 8 mL to 15 mL per 1 g of fresh Rhizoma dioscoreae to the Rhizoma dioscoreae fermentation broth to conduct the hot water extraction.

Preferably, the hot water extraction is conducted at 80° C. to 90° C.

Preferably, the hot water extraction is conducted for 2 h to 5 h.

Preferably, the concentrating is conducted to a volume ⅙ to ⅓ of an original volume.

Preferably, the method further comprises deproteinizing the concentrated solution by Sevag method, subjecting to organic reagent removal through rotary evaporation, and then subjecting to dialysis in water using a dialysis bag with a molecular weight cut-off of 7,000 Da to 9,000 Da for 36 h to 60 h to obtain a dialysate, drying the dialysate to produce the Rhizoma dioscoreae polysaccharide.

The present disclosure also provides a highly-active Rhizoma dioscoreae polysaccharide produced by the preparation method.

The present disclosure also provides use of the highly-active Rhizoma dioscoreae polysaccharide or a highly-active Rhizoma dioscoreae polysaccharide produced by the preparation method in preparation of a product for inhibiting an activity of lipase and/or amylase.

The present disclosure also provides use of the highly-active Rhizoma dioscoreae polysaccharide or a highly-active Rhizoma dioscoreae polysaccharide produced by the preparation method in preparation of a product for consumption by an obesity patient or a hyperglycemia patient.

Compared with the prior art, the present disclosure has the following beneficial effects:
(1) The method for preparing the Rhizoma dioscoreae polysaccharide in the present disclosure is simple and cost-effective, and may be easily used for large-scale production.
(2) The method for preparing the Rhizoma dioscoreae polysaccharide in the present disclosure adopts a combination of ultra-high pressure and lactic acid bacteria-based fermentation treatments. Compared to the existing preparation methods, the preparation method of the present disclosure significantly enhances the extraction yield of the Rhizoma dioscoreae polysaccharide.
(3) The Rhizoma dioscoreae polysaccharide prepared by the present disclosure demonstrates a more significant inhibitory effect on lipase and amylase activities than Rhizoma dioscoreae polysaccharides extracted by the conventional methods. The Rhizoma dioscoreae polysaccharide of the present disclosure is suitable for consumption by individuals with obesity, hyperglycemia, or diabetes. Moreover, the Rhizoma dioscoreae polysaccharide exhibits superior efficacy in promoting the proliferation and colonization of probiotics, and may be used for developing Rhizoma dioscoreae-based functional foods. Thus, the present disclosure holds considerable importance for the deep processing of Rhizoma dioscoreae.

### BRIEF DESCRIPTION OF THE DRAWINGS

FIG. 1 shows results of the screening of fermentation strains;

FIGS. 2A-2C show results of single-factor experiments for an ultra-high pressure treatment; and

FIGS. 3A-3C show results of single-factor experiments for fermentation.

### DETAILED DESCRIPTION OF THE EMBODIMENTS

The present disclosure provides a method for preparing a highly-active Rhizoma dioscoreae polysaccharide, including the following steps: A Rhizoma dioscoreae slurry is subjected to an ultra-high pressure treatment to produce an ultra-high pressure-treated Rhizoma dioscoreae slurry. Lactiplantibacillus plantarum subsp. plantarum is inoculated into the ultra-high pressure-treated Rhizoma dioscoreae slurry, and fermentation is conducted to produce a Rhizoma dioscoreae fermentation broth. The Rhizoma dioscoreae fermentation broth is subjected to hot water extraction and then centrifugation to obtain a supernatant, the supernatant is concentrated to obtain a concentrated solution, and the concentrated solution is dried to produce the Rhizoma dioscoreae polysaccharide.

In the present disclosure, a mass-to-volume ratio of fresh Rhizoma dioscoreae to water in the Rhizoma dioscoreae slurry is 1:4 to 1:8 (final material-to-liquid ratio). In the present disclosure, fresh Rhizoma dioscoreae is peeled, and then peeled fresh Rhizoma dioscoreae and water are mixed in a mass ratio of 1:0.5-1.5 and beaten to produce an preliminary Rhizoma dioscoreae slurry. Water is then added to the preliminary Rhizoma dioscoreae slurry until a mass-to-volume ratio of fresh Rhizoma dioscoreae to water in a resulting system is 1:4 to 1:8, and thorough mixing is conducted to produce the Rhizoma dioscoreae slurry. In the present disclosure, to ensure a basically-consistent particle size for Rhizoma dioscoreae, fresh Rhizoma dioscoreae is first beaten according to the specific ratio, and then thoroughly mixed with a corresponding amount of water according to the final ratio to produce the Rhizoma dioscoreae slurry for subsequent experiments.

In the present disclosure, when the Rhizoma dioscoreae slurry is subjected to the ultra-high pressure treatment, a cell wall structure may be disrupted under an ultra-high pressure, such that intracellular polysaccharides may be easily dissolved. Moreover, the ultra-high pressure may alter a crystalline structure of starch, thereby changing the gelatinization properties, enhancing the solubility, and reducing the viscosity. However, factors such as an ultra-high pressure intensity, a treatment time, and a material-to-liquid ratio may affect the extraction efficiency of the Rhizoma dioscoreae polysaccharide. A too-high pressure will severely disrupt the cell wall of Rhizoma dioscoreae and promote the dissolution of impurity substances, resulting in a reduced purity and yield of the extracted polysaccharide. A too-low pressure will lead to limited disruption for the cell wall, which hinders the release of the polysaccharide and reduces the yield. If the pressure-holding time is too short, the polysaccharide dissolution will be insufficient, resulting in suboptimal extraction efficiency. If the pressure-holding time is too long, the prolonged pressure-holding time will not further increase the yield because the polysaccharide has been completely released, but will increase the energy consumption. Therefore, in the present disclosure, the ultra-high pressure treatment is conducted under a pressure of 200 MPa to 600 MPa with a pressure-holding time of 5 min to 11 min. Preferably, the ultra-high pressure treatment is conducted under a pressure of 300 MPa to 500 MPa with a pressure-holding time of 6 min to 10 min.

In the present disclosure, the ultra-high pressure-treated Rhizoma dioscoreae slurry needs to be sterilized. The sterilization in the present disclosure is intended to eliminate various microbial strains in the ultra-high pressure-treated Rhizoma dioscoreae slurry and achieve the gelatinization for Rhizoma dioscoreae starch. A mode for the sterilization in the present disclosure is determined by comparing the conventional high-temperature sterilization with the pasteurization. The conventional high-temperature sterilization (121° C., 15 min) will alter a structure of the Rhizoma dioscoreae polysaccharide. A high temperature of the conventional high-temperature sterilization will cause the degradation of Rhizoma dioscoreae polysaccharide chains, thereby reducing the bioactivity. The pasteurization is conducted at 60° C. to 80° C. (most preferably 70° C.) for 20 min to 40 min (most preferably 30 min). Thus, the pasteurization may effectively eliminate contaminating microorganisms in the Rhizoma dioscoreae slurry without altering the structure and bioactivity of the active Rhizoma dioscoreae polysaccharide, and may also make the starch in Rhizoma dioscoreae gelatinized, thereby enhancing the utilization of the starch by lactic acid bacteria.

The Lactiplantibacillus plantarum subsp. plantarum adopted in the present disclosure is determined through experimental screening. According to the comparative analysis of different bacterial strains, the fermentation of the Rhizoma dioscoreae slurry with the Lactiplantibacillus plantarum subsp. plantarum leads to the highest yield of the Rhizoma dioscoreae polysaccharide. In an embodiment of the present disclosure, the Lactiplantibacillus plantarum subsp. plantarum is purchased from the Guangdong Microbial Culture Collection Center (GDMCC), with an accession number of GIM 1.380. An inoculum size of the Lactiplantibacillus plantarum subsp. plantarum in the present disclosure is highly correlated with a growth state of the Lactiplantibacillus plantarum subsp. plantarum, and affects a fermentation time. If the inoculum size is too small, a too-long fermentation time will be required. If the inoculum size is too large, lactic acid bacteria will excessively grow, and will secrete carbohydrases to degrade polysaccharides, which reduces the polysaccharide yield. Therefore, the inoculum size of the Lactiplantibacillus plantarum subsp. plantarum in the present disclosure (expressed as a percentage of a mass of the fresh Rhizoma dioscoreae to a volume of a suspension of the Lactiplantibacillus plantarum subsp. plantarum) is 2% to 6% and preferably 3% to 5%. The fermentation is conducted at 35° C. to 40° C. and preferably 37° C. The fermentation is conducted for 1 d to 4 d and preferably 1.5 d to 2.5 d. In the present disclosure, a concentration of the Lactiplantibacillus plantarum subsp. plantarum is 0.5×10⁹ cfu/mL to 1.5×10⁹ cfu/mL and preferably 1×10⁹ cfu/mL. A method for producing the suspension of the Lactiplantibacillus plantarum subsp. plantarum in the present disclosure includes the following steps: Lactiplantibacillus plantarum subsp. plantarum strain stored in liquid nitrogen is taken and coated on an MRS agar medium by a plate streaking method, and cultured for 48 h. Single colonies are then picked and inoculated into 10 mL of MRS broth, cultured for 12 h, and centrifuged (3,000 g, 10 min) to produce a first supernatant and a first pellet. The first supernatant is discarded. The first pellet is resuspended in 10 mL of MRS broth, cultured for 12 h, and centrifuged (3,000 g, 10 min) to produce a second supernatant and a second pellet. The second supernatant is discarded. The second pellet is resuspended evenly in 5 mL of normal saline (0.9% NaCl) to produce the suspension of the Lactiplantibacillus plantarum subsp. plantarum.

In the present disclosure, water is added to the Rhizoma dioscoreae fermentation broth according to a final material-to-liquid ratio of 8 mL/g to 12 mL/g, and a resulting mixture is subjected to extraction in hot water at 80° C. to 90° C. for 2 h to 5 h and then centrifuged at 3,000 r/min to 5,000 r/min. A resulting supernatant is collected, and concentrated to a volume ⅙ to ⅓ of an original volume. A resulting concentrate is deproteinized by Sevag method (a Sevag reagent is added in a volume ¼ of a volume of the concentrate, and shaking is conducted vigorously for 30 min. The Sevag reagent is prepared by mixing chloroform and n-butanol at 4:1), subjected to organic reagent removal through rotary evaporation, and then subjected to dialysis in pure water for 40 h to 55 h using a dialysis bag with a molecular weight cut-off of 7,500 Da to 8,500 Da. A resulting dialysate is collected and dried to produce the Rhizoma dioscoreae polysaccharide. The hot water extraction in the present disclosure is preferably conducted at 80° C. for 3 h. In the hot water extraction of the present disclosure, a too-high heating temperature may lead to destruction of polysaccharide components, and a too-low heating temperature may result in insufficient extraction. In the present disclosure, the concentrating is preferably conducted to a volume ⅕ of the original volume.

The present disclosure also provides a highly-active Rhizoma dioscoreae polysaccharide produced by the preparation method.

The present disclosure also provides a use of the highly-active Rhizoma dioscoreae polysaccharide or a highly-active Rhizoma dioscoreae polysaccharide produced by the preparation method in preparation of a product for inhibiting activities of lipase and amylase. The inhibition of pancreatic lipase in the present disclosure has long been utilized as a therapeutic target for obesity intervention. α-amylase inhibitors may delay the digestion of carbohydrates in hyperglycemia or diabetes patients, thereby reducing an absorption rate of glucose and attenuating postprandial blood glucose elevation. The highly-active Rhizoma dioscoreae polysaccharide prepared in the present disclosure may serve as an inhibitor for both lipase and amylase, and demonstrates a specified preventive and/or therapeutic effect for obesity, hyperglycemia, and diabetes patients. The Rhizoma dioscoreae polysaccharide in the present disclosure may also be used for promoting the proliferation and colonization of probiotics.

In the present disclosure, unless otherwise specified, all components or reagents are commercially-available products well known to those skilled in the art.

The technical solutions in the present disclosure will be clearly and completely described below with reference to examples of the present disclosure. Apparently, the described examples are merely some rather than all of the examples of the present disclosure. All other examples obtained by a person of ordinary skill in the art based on the examples of the present disclosure without creative efforts shall fall within the protection scope of the present disclosure.

### Example 1

In this example, a method for preparing a highly-active Rhizoma dioscoreae polysaccharide was provided, including the following steps:
1) Ultra-high pressure treatment: 100 g of fresh Rhizoma dioscoreae was taken, mixed with water at a mass ratio of 1:1, and beaten to produce an preliminary Rhizoma dioscoreae slurry. Then, water was added to the preliminary Rhizoma dioscoreae slurry until a ratio of fresh Rhizoma dioscoreae to water in a resulting system was 1:5 (W/V, g/mL), and thorough mixing was conducted to produce a Rhizoma dioscoreae slurry. The Rhizoma dioscoreae slurry was placed in a polyethylene bag, sealed, and subjected to the ultra-high pressure treatment under a pressure of 500 MPa with a pressure-holding time of 10 min to produce an ultra-high pressure-treated Rhizoma dioscoreae slurry.
2) Lactobacillus-based fermentation: A Lactiplantibacillus plantarum subsp. plantarum strain (GIM 1.380) stored in liquid nitrogen was taken, coated on an MRS agar medium by a plate streaking method, and cultured for 48 h. Single colonies were then picked and inoculated into 10 mL of MRS broth, cultured for 12 h, and centrifuged (3,000 g, 10 min) to produce a first supernatant and a first pellet. The first supernatant was discarded. The first pellet was resuspended in 10 mL of MRS broth, cultured for 12 h, and centrifuged (3,000 g, 10 min) to produce a second supernatant and a second pellet. The second supernatant was discarded. The second pellet was resuspended evenly in 5 mL of normal saline (0.9% NaCl) to produce a Lactiplantibacillus plantarum subsp. plantarum suspension at a concentration of 1× 10⁹ cfu/mL.

The ultra-high pressure-treated Rhizoma dioscoreae slurry was sterilized at 80° C. for 30 min, and then the Lactiplantibacillus plantarum subsp. plantarum suspension was inoculated at an inoculum size of 5% (5 mL of the Lactiplantibacillus plantarum subsp. plantarum suspension was inoculated per 100 g of the fresh Rhizoma dioscoreae). Fermentation was conducted for 2 d at 37° C. to produce a Rhizoma dioscoreae fermentation broth.
3) Extraction: Water was added to the Rhizoma dioscoreae fermentation broth according to a final material-to-liquid ratio of 10 mL/g (10 ml of water per 1 g of the fresh Rhizoma dioscoreae), and a resulting mixture was subjected to extraction in hot water at 80° C. for 3 h and then centrifuged at 4,000 r/min. A resulting supernatant was collected, and concentrated to a volume ⅕ of an original volume. A resulting concentrate was deproteinized by Sevag method (a Sevag reagent was added in a volume ¼ of a volume of the concentrate, and shaking was conducted vigorously for 30 min. The Sevag reagent was prepared by mixing chloroform and n-butanol at 4:1), subjected to organic reagent removal through rotary evaporation, and then subjected to dialysis in pure water for 48 h using a dialysis bag with a molecular weight cut-off of 8,000 Da. A resulting dialysate was collected and dried to produce the Rhizoma dioscoreae polysaccharide.

### Example 2

In this example, a method for preparing a highly-active Rhizoma dioscoreae polysaccharide was provided, including the following steps:
1) Ultra-high pressure treatment: 100 g of fresh Rhizoma dioscoreae was taken, mixed with water at a mass ratio of 1:1, and beaten to produce an preliminary Rhizoma dioscoreae slurry. Then, water was added to the preliminary Rhizoma dioscoreae slurry until a ratio of fresh Rhizoma dioscoreae to water in a resulting system was 1:7 (g/mL), and thorough mixing was conducted to produce a Rhizoma dioscoreae slurry. The Rhizoma dioscoreae slurry was placed in a polyethylene bag, sealed, and subjected to the ultra-high pressure treatment under a pressure of 300 MPa with a pressure-holding time of 8 min to produce an ultra-high pressure-treated Rhizoma dioscoreae slurry.
2) Lactobacillus-based fermentation: The preparation of a Lactiplantibacillus plantarum subsp. plantarum suspension and the selection of a strain and a concentration thereof were all the same as those in Example 1.

The ultra-high pressure-treated Rhizoma dioscoreae slurry was sterilized at 80° C. for 30 min, and then the Lactiplantibacillus plantarum subsp. plantarum suspension was inoculated at an inoculum size of 5% (5 mL of the Lactiplantibacillus plantarum subsp. plantarum suspension was inoculated per 100 g of the fresh Rhizoma dioscoreae). Fermentation was conducted for 3 d at 37° C. to produce a Rhizoma dioscoreae fermentation broth.
3) Extraction: Water was added to the Rhizoma dioscoreae fermentation broth according to a final material-to-liquid ratio of 10 mL/g (10 ml of water per 1 g of the fresh Rhizoma dioscoreae), and a resulting mixture was subjected to extraction in hot water at 85° C. for 3 h and then centrifuged at 4,000 r/min. A resulting supernatant was collected, and concentrated to a volume ⅕ of an original volume. A resulting concentrate was deproteinized by Sevag method (a Sevag reagent was added in a volume ¼ of a volume of the concentrate, and shaking was conducted vigorously for 30 min. The Sevag reagent was prepared by mixing chloroform and n-butanol at 4:1), subjected to organic reagent removal through rotary evaporation, and then subjected to dialysis in pure water for 48 h using a dialysis bag with a molecular weight cut-off of 8,000 Da. A resulting dialysate was collected and dried to produce the Rhizoma dioscoreae polysaccharide.

### Example 3

In this example, a method for preparing a highly-active Rhizoma dioscoreae polysaccharide was provided, including the following steps:
1) Ultra-high pressure treatment: 100 g of fresh Rhizoma dioscoreae was taken, mixed with water at a mass ratio of 1:1, and beaten to produce an preliminary Rhizoma dioscoreae slurry. Then, water was added to the preliminary Rhizoma dioscoreae slurry until a ratio of fresh Rhizoma dioscoreae to water in a resulting system was 1:7 (g/mL), and thorough mixing was conducted to produce a Rhizoma dioscoreae slurry. The Rhizoma dioscoreae slurry was placed in a polyethylene bag, sealed, and subjected to the ultra-high pressure treatment under a pressure of 500 MPa with a pressure-holding time of 6 min to produce an ultra-high pressure-treated Rhizoma dioscoreae slurry.
2) Lactobacillus-based fermentation: The preparation of a Lactiplantibacillus plantarum subsp. plantarum suspension and the selection of a strain and a concentration thereof were all the same as those in Example 1.

The ultra-high pressure-treated Rhizoma dioscoreae slurry was sterilized at 80° C. for 30 min, and then the Lactiplantibacillus plantarum subsp. plantarum suspension was inoculated at an inoculum size of 3% (3 mL of the Lactiplantibacillus plantarum subsp. plantarum suspension was inoculated per 100 g of the fresh Rhizoma dioscoreae). Fermentation was conducted for 2 d at 37° C. to produce a Rhizoma dioscorede fermentation broth.
3) Extraction: Water was added to the Rhizoma dioscoreae fermentation broth according to a final material-to-liquid ratio of 10 mL/g (10 mL of water per 1 g of the fresh Rhizoma dioscoreae), and a resulting mixture was subjected to extraction in hot water at 90° C. for 3 h and then centrifuged at 4,000 r/min. A resulting supernatant was collected, and concentrated to a volume ⅕ of an original volume. A resulting concentrate was deproteinized by Sevag method (a Sevag reagent was added in a volume ¼ of a volume of the concentrate, and shaking was conducted vigorously for 30 min. The Sevag reagent was prepared by mixing chloroform and n-butanol at 4:1), subjected to organic reagent removal through rotary evaporation, and then subjected to dialysis in pure water for 48 h using a dialysis bag with a molecular weight cut-off of 8,000 Da. A resulting dialysate was collected and dried to produce the Rhizoma dioscoreae polysaccharide.

### Example 4

In this example, a method for preparing a highly-active Rhizoma dioscoreae polysaccharide was provided, including the following steps:
1) Ultra-high pressure treatment: 100 g of fresh Rhizoma dioscoreae was taken, mixed with water at a mass ratio of 1:1, and beaten to produce an preliminary Rhizoma dioscoreae slurry. Then, water was added to the preliminary Rhizoma dioscoreae slurry until a ratio of fresh Rhizoma dioscoreae to water in a resulting system was 1:7 (g/mL), and thorough mixing was conducted to produce a Rhizoma dioscoreae slurry. The Rhizoma dioscoreae slurry was placed in a polyethylene bag, sealed, and subjected to the ultra-high pressure treatment under a pressure of 400 MPa with a pressure-holding time of 6 min to produce an ultra-high pressure-treated Rhizoma dioscoreae slurry.
2) Lactobacillus-based fermentation: The preparation of a Lactiplantibacillus plantarum subsp. plantarum suspension and the selection of a strain and a concentration thereof were all the same as those in Example 1.

The ultra-high pressure-treated Rhizoma dioscoreae slurry was sterilized at 80° C. for 30 min, and then the Lactiplantibacillus plantarum subsp. plantarum suspension was inoculated at an inoculum size of 5% (5 mL of the Lactiplantibacillus plantarum subsp. plantarum suspension was inoculated per 100 g of the fresh Rhizoma dioscoreae). Fermentation was conducted for 3 d at 37° C. to produce a Rhizoma dioscoreae fermentation broth.
3) Extraction: Water was added to the Rhizoma dioscoreae fermentation broth according to a final material-to-liquid ratio of 10 mL/g (10 mL of water per 1 g of the fresh Rhizoma dioscoreae), and a resulting mixture was subjected to extraction in hot water at 90° C. for 3 h and then centrifuged at 4,000 r/min. A resulting supernatant was collected, and concentrated to a volume ⅕ of an original volume. A resulting concentrate was deproteinized by Sevag method (a Sevag reagent was added in a volume ¼ of a volume of the concentrate, and shaking was conducted vigorously for 30 min. The Sevag reagent was prepared by mixing chloroform and n-butanol at 4:1), subjected to organic reagent removal through rotary evaporation, and then subjected to dialysis in pure water for 48 h using a dialysis bag with a molecular weight cut-off of 8,000 Da. A resulting dialysate was collected and dried to produce the Rhizoma dioscoreae polysaccharide.

Test method 1: The Rhizoma dioscoreae polysaccharides prepared in Examples 1 to 4 were each tested for a polysaccharide content, and polysaccharide yields were calculated. A specific method was as follows:

Determination of a total sugar content: A total sugar content in an extract was determined by a phenol-sulfuric acid method with glucose as a standard. The principle of the total sugar content determination is as follows: At a high temperature, sulfuric acid decomposes a polysaccharide into a monosaccharide, the monosaccharide is promptly dehydrated to produce a furfural derivative, and the furfural derivative is combined with phenol to produce an orange-yellow complex. The orange-yellow complex has a maximum absorbance value at 490 nm, and there is a linear relationship in a specific range. Glucose (oven-dried at 105° C. to a constant weight before use) was prepared into standard solutions with varying concentrations (0.02 mg/mL, 0.04 mg/mL, 0.06 mg/mL, 0.08 mg/mL, and 0.1 mg/mL). A concentration of a polysaccharide sample was 0.1 mg/mL. 1 mL of a sample to be tested was taken, 1 mL of a phenol solution at a concentration of 5% was added, and then 5 mL of concentrated sulfuric acid was immediately added. A resulting mixture was thoroughly mixed, allowed to stand for 30 min, and then tested for absorbance at 490 nm. A standard curve was plotted with a mass concentration of glucose as an x-coordinate and an absorbance value as a y-coordinate. A total sugar content in a polysaccharide sample was calculated according to the standard curve.

Determination of a reducing sugar content: A reducing sugar content in an extract was determined by a 3,5-dinitrosalicylic acid (DNS) method with glucose as a standard. The principle of the DNS method is as follows: DNS undergoes an oxidation-reduction reaction with a reducing sugar to produce 3-amino-5-nitrosalicylic acid. 3-amino-5-nitrosalicylic acid has a maximum absorbance value at 540 nm, and there is a linear relationship in a specific range. Preparation of a DNS reagent: 6.3 g of DNS and 262 mL of 2 mol/L sodium hydroxide were added to 500 mL of a hot aqueous solution including 185 g of potassium sodium tartrate, and then, 5 g of phenol and 5 g of sodium sulfite were added. A resulting mixture was stirred to allow complete dissolution, then cooled, and diluted with water to 1,000 mL to produce the DNS reagent, which was stored in a brown bottle at 4° C. for later use. Glucose (oven-dried at 105° C. to a constant weight before use) was prepared into standard solutions with varying concentrations (0.2 mg/mL, 0.4 mg/mL, 0.6 mg/mL, 0.8 mg/mL, and 1 mg/mL). A concentration of a polysaccharide sample was 5 mg/mL. 1 mL of a sample to be tested was taken, and 0.75 mL of the DNS reagent was added to produce a first mixed system. The first mixed system was fully shaken, heated in a boiling-water bath for 5 min, and then rapidly cooled in an ice-water bath to room temperature. 10.75 mL of distilled water was added to produce a second mixed system. The second mixed system was vortexed for thorough mixing, and then tested for absorbance at 540 nm. A standard curve was plotted with a mass concentration of glucose as an x-coordinate and an absorbance value as a y-coordinate. A total sugar content in a polysaccharide sample was calculated according to the standard curve.

$$Polysaccharide content = total sugar content - reducing sugar ontent .$$

$$Extraction yield = polysaccharide content / mass of fresh Rhizoma \\ \\ Dioscoreae \times 100 \% .$$

TABLE 1
Extraction yields of the Rhizoma Dioscoreae
polysaccharides prepared in Examples 1 to 4

| Process parameter | Example 1 | Example 2 | Example 3 | Example 4 |
|---|---|---|---|---|
| Material-to-liquid ratio (g/mL) | 1:5 | 1:7 | 1:7 | 1:7 |
| Pressure (MPa) | 500 | 300 | 500 | 400 |
| Pressure-holding time (min) | 10 | 8 | 6 | 6 |
| Inoculum size (%) | 5 | 5 | 3 | 5 |
| Fermentation time (d) | 2 | 3 | 2 | 3 |
| Polysaccharide extraction yield (%) | 10.26 | 8.38 | 9.17 | 7.96 |

### Example 5 Process Optimization for the Method for Preparing the Rhizoma dioscoreae Polysaccharide

1. Strain screening: A total of 8 different strains and a strain combination were selected for the fermentation of Rhizoma dioscoreae, including: Lactobacillus fermentum GIM 1.1796, Lactobacillus acidophilus GIM 1.731, Lactiplantibacillus plantarum subsp. plantarum GIM 1.380, Lactobacillus casei GIM 1.410, Bacillus subtilis GIM 1.372, Aspergillus niger GIM 3.576, Rhizopus oryzae GIM 3.127, Trichoderma viride GIM 3.443, and Lactiplantibacillus plantarum subsp. plantarum GIM 1.380+Bacillus subtilis GIM 1.372. All of the above strains were purchased from the Guangdong Microbial Culture Collection Center (GDMCC). Specific experimental steps were the same as those in Example 1, except that: In a mold stain-related experiment, a final material-to-liquid ratio of a Rhizoma dioscoreae slurry to water in the step (1) was 1:2, an inoculum size was 1%, and an inoculation concentration was approximately 1×10⁶ CFU/mL. In a Lactiplantibacillus plantarum subsp. plantarum GIM 1.380+Bacillus subtilis GIM 1.372 combination-related experiment, a volume ratio of a Lactiplantibacillus plantarum subsp. plantarum suspension to a Bacillus subtilis suspension in a mixed stain suspension was 1:1

A Lactobacillus suspension was prepared by the same method as in Example 1.

Preparation of a mold suspension: A preserved strain (Aspergillus niger GIM 3.576, Rhizopus oryzae GIM 3.127, and Trichoderma viride GIM 3.443) was coated on a potato dextrose agar (PDA) plate and cultured at 30° C. for 5 d to 7 d until the PDA plate was fully covered with mycelia. Then, 10 mL of sterile water was added, and the mycelia on the agar surface were carefully scraped off, transferred to a sterilized centrifuge tube, vortexed for thorough mixing, and filtered through a nucleic acid purification column with a sterilized absorbent cotton. A resulting filtrate was collected and transferred to a 50 mL centrifuge tube to produce a spore suspension for later use.

Preparation of a Bacillus subtilis suspension: A Bacillus subtilis strain (GIM 1.372) stored in liquid nitrogen was taken, coated on an LB agar medium by a plate streaking method, and cultured for 48 h. Single colonies were then picked and inoculated into 10 mL of LB broth, cultured for 12 h, and centrifuged (3,000 g, 10 min) to produce a first supernatant and a first pellet. The first supernatant was discarded. The first pellet was resuspended in 10 mL of LB broth, cultured for 12 h, and centrifuged (3,000 g, 10 min) to produce a second supernatant and a second pellet. The second supernatant was discarded. The second pellet was resuspended evenly in 5 mL of normal saline (0.9% NaCl) to produce a Bacillus subtilis suspension.

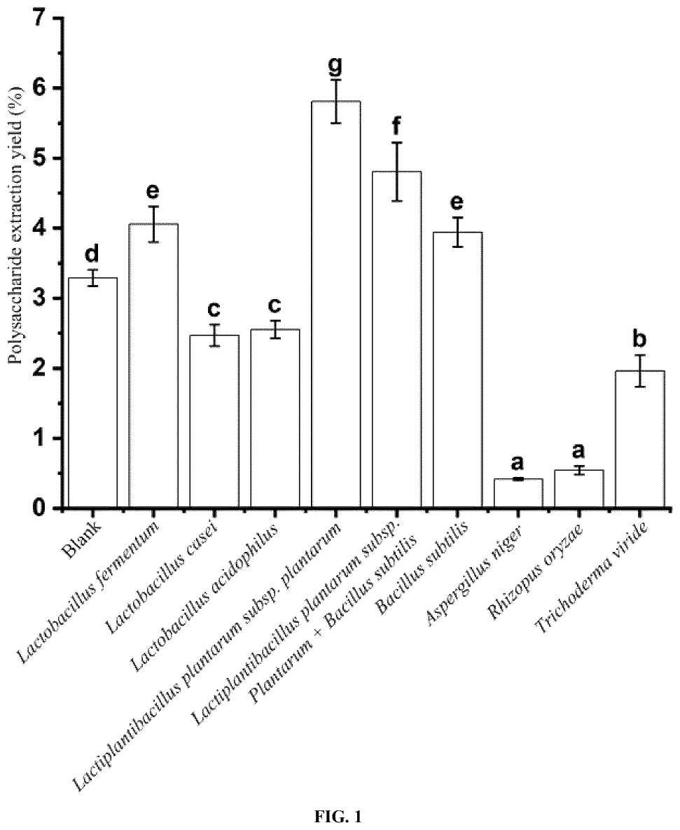

Results were shown in FIG. 1. The Lactiplantibacillus plantarum subsp. plantarum (GIM 1.380) could significantly enhance the yield of the Rhizoma dioscoreae polysaccharide compared to other strains.

### 2. Single-Factor Experiments for the Ultra-High Pressure Treatment

1) Optimization of a material-to-liquid ratio (Rhizoma dioscoreae:water, W:V): Steps of the ultra-high pressure treatment were the same as the steps of the ultra-high pressure treatment in Example 1, except that, the material-to-liquid ratio was set to 1:2, 1:3, 1:4, 1:5, and 1:6, a pressure was set to 400 MPa, a treatment time was set to 8 min, and 20 g of fresh Rhizoma dioscoreae was adopted.
2) Optimization of a pressure: Steps of the ultra-high pressure treatment were the same as the steps of the ultra-high pressure treatment in Example 1, except that, a pressure was set to 200 MPa, 300 MPa, 400 MPa, 500 MPa, and 600 MPa, a pressure-holding time was set to 8 min, a molar material-to-liquid ratio was set to 1:4, and 20 g of fresh Rhizoma dioscoreae was adopted.
3) Optimization of a pressure-holding time: Steps of the ultra-high pressure treatment were the same as the steps of the ultra-high pressure treatment in Example 1, except that, the pressure-holding time was set to 2 min, 4 min, 6 min, 8 min, and 10 min, a pressure was set to 400 MPa, a material-to-liquid ratio was set to 1:4, and 20 g of fresh Rhizoma dioscoreae was adopted.

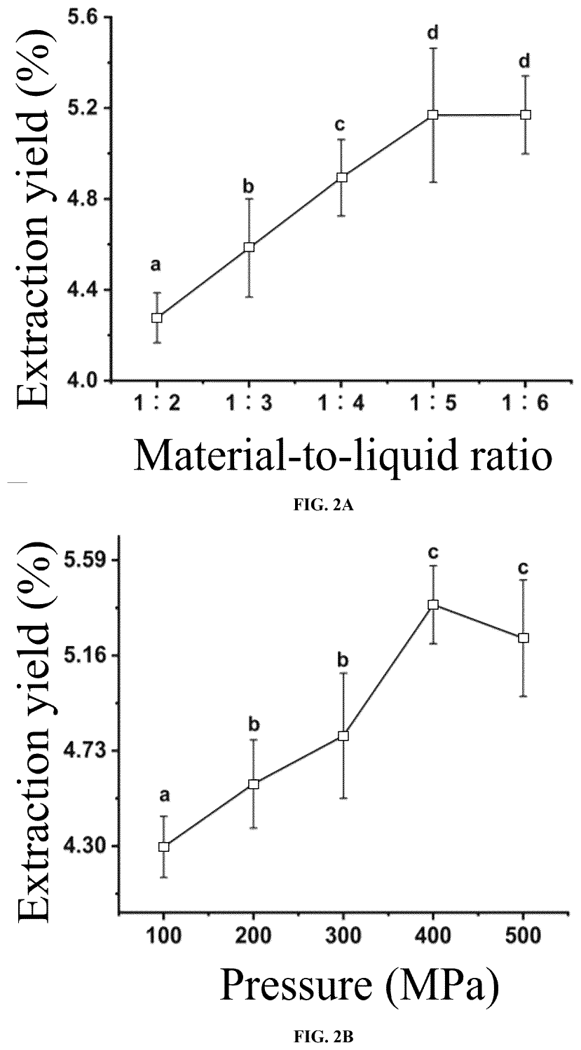

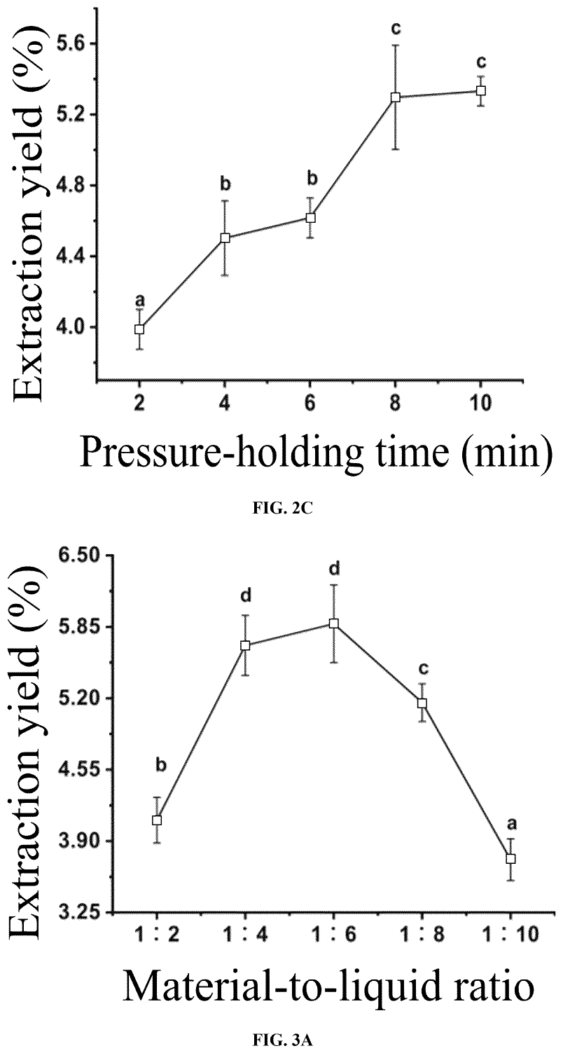

Polysaccharide extraction: An ultra-high pressure-treated sample (including 20 g of fresh Rhizoma dioscoreae) was taken, and a corresponding amount of distilled water was added according to a material-to-liquid ratio of 1:10. Extraction was conducted in an 80° C. water bath for 3 h, and centrifugation was then conducted at 4,000 rpm. A resulting supernatant was collected, concentrated, and diluted to 20 mL. A four-fold volume of 95% ethanol was added, and a resulting system was allowed to stand overnight at 4° C., and then centrifuged. A resulting precipitate was collected, redissolved, diluted to 100 mL, and tested by a phenol-sulfuric acid method and a DNS method. A difference between values determined by these two methods was approximately equal to a polysaccharide content. A polysaccharide extraction yield was calculated as a ratio of a polysaccharide content to a mass of fresh Rhizoma dioscoreae. Results were shown in FIGS. 2A-2C.

As shown in FIGS. 2A-2C, in the ultra-high pressure treatment process, a polysaccharide extraction yield increased as the material-to-liquid ratio, the pressure, and the pressure-holding time increased within specified ranges. In the single-factor experiments, the material-to-liquid ratio of 1:5, the pressure of 400 MPa, and the pressure-holding time of 10 min led to the highest polysaccharide extraction yields.

### 3. Single-Factor Experiments for Fermentation

1) Optimization of a material-to-liquid ratio (Rhizoma dioscoreae:water, W:V): The material-to-liquid ratio was set to 1:2, 1:4, 1:6, 1:8, and 1:10. An inoculum size was set to 2%. A fermentation time was set to 2 d.
2) Optimization of an inoculum size: The inoculum size (based on a mass of Rhizoma dioscoreae, with a microbial concentration of 1×10⁹ cfu/mL) was set to 1%, 2%, 3%, 4%, and 5%. A fermentation time was set to 2 d. A material-to-liquid ratio was set to 1:4.
3) Optimization of a fermentation time: The fermentation time was set to 1 d, 2 d, 3 d, 4 d, and 5 d. An inoculum size was set to 2%. A material-to-liquid ratio was set to 1:4.

Fermentation steps for Rhizoma dioscoreae under the process parameters set above were as follows: Rhizoma dioscoreae was beaten according to a ratio of 1:1 (Rhizoma dioscorede:water, W:V). 20 g of a resulting Rhizoma dioscoreae slurry was weighed and transferred into a 150 ml conical flask. A specified amount of water was added according to different material-to-liquid ratios, and the conical flask was sealed and placed in an autoclave for pasteurization (80° C., 30 min). Lactiplantibacillus plantarum subsp. plantarum (GIM 1.380) was inoculated at varying inoculum sizes. Fermentation was conducted at 37° C. with different fermentation durations.

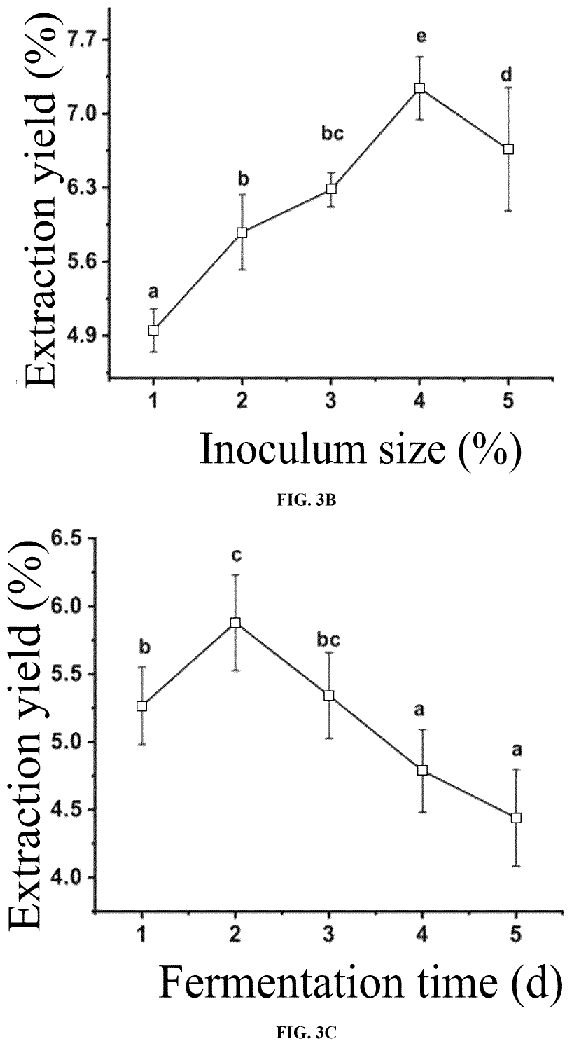

Extraction and determination of polysaccharides: After the fermentation was completed, a sample was collected and tested by the same methods as in the step 2 of this example. Results were shown in FIGS. 3A-3C.

As shown in FIGS. 3A-3C, in the single-factor experiments for fermentation, the polysaccharide extraction yield initially increased and then decreased as the material-to-liquid ratio, the inoculum size, and the fermentation time increased. The highest polysaccharide extraction yields corresponded to the following factor conditions, respectively:material-to-liquid ratio: 1:6, inoculum size: 4%, and fermentation time: 2 d.

### 4. Orthogonal Experiment

The extraction process for the Rhizoma dioscoreae polysaccharide was optimized through an orthogonal experiment with five factors and three levels. Experimental process parameters and results were shown in Tables 2 and 3.

TABLE 2
Comparison of polysaccharides produced from different extraction processes

| | Column No. | | | | | |
|---|---|---|---|---|---|---|
| Experiment | Inoculum | Fermentation | Material-to-liquid | Pressure | Pressure-holding | Polysaccharide |
| No. | size (%) | time (d) | ratio | (MPa) | time (min) | extraction yield (%) |
| 1 | 3 | 1 | 1:5 | 300 | 6 | 4.59 |
| 2 | 3 | 2 | 1:6 | 400 | 8 | 4.91 |
| 3 | 3 | 3 | 1:7 | 500 | 10 | 6.74 |
| 4 | 4 | 1 | 1:7 | 400 | 8 | 4.80 |
| 5 | 4 | 2 | 1:5 | 500 | 10 | 7.32 |
| 6 | 4 | 3 | 1:6 | 300 | 6 | 5.74 |
| 7 | 5 | 1 | 1:6 | 500 | 6 | 6.40 |
| 8 | 5 | 2 | 1:7 | 300 | 8 | 8.38 |
| 9 | 5 | 3 | 1:5 | 400 | 10 | 6.02 |
| 10 | 3 | 1 | 1:6 | 400 | 10 | 3.88 |
| 11 | 3 | 2 | 1:7 | 500 | 6 | 9.17 |
| 12 | 3 | 3 | 1:5 | 300 | 8 | 4.63 |
| 13 | 4 | 1 | 1:7 | 300 | 10 | 5.89 |
| 14 | 4 | 2 | 1:5 | 400 | 6 | 5.68 |
| 15 | 4 | 3 | 1:6 | 500 | 8 | 6.17 |
| 16 | 5 | 1 | 1:5 | 500 | 8 | 7.63 |
| 17 | 5 | 2 | 1:6 | 300 | 10 | 6.13 |
| 18 | 5 | 3 | 1:7 | 400 | 6 | 7.96 |
| K₁ | 33.92 | 33.18 | 39.54 | 35.36 | 35.88 | |
| K₂ | 35.60 | 41.59 | 36.51 | 33.25 | 33.21 | |
| K₃ | 42.52 | 37.26 | 35.99 | 43.44 | 42.95 | |
| R | 2.87 | 2.80 | 1.19 | 3.40 | 3.24 | |

TABLE 3
Results of tests of between-subjects
effects in the orthogonal experiment

| | Tests of between-subjects effects | | | | |
|---|---|---|---|---|---|
| Dependent variable: 8 | Type III sum | | Mean | | |
| Source | of squares | df | square | F | Sig. |
| Adjusted model | 32.117 | 10 | 3.212 | 7.764 | 0.006 |
| Intercept | 697.361 | 1 | 697.361 | 1685.887 | 0.000 |
| Inoculum size | 6.924 | 2 | 3.462 | 8.370 | 0.014 |
| Fermentation time | 5.893 | 2 | 2.946 | 7.123 | 0.021 |
| Material-to-liquid ratio | 1.228 | 2 | 0.614 | 1.484 | 0.290 |
| Pressure | 9.642 | 2 | 4.821 | 11.654 | 0.006 |
| Pressure-holding time | 8.430 | 2 | 4.215 | 10.190 | 0.008 |
| Error | 2.896 | 7 | 0.414 | | |
| Total | 732.374 | 18 | | | |
| Corrected total | 35.012 | 17 | | | |

According to the results in Tables 2 and 3, the optimal process is as follows: inoculum size: 5%, fermentation time: 2 d, material-to-liquid ratio: 1:5, pressure: 500 MPa, and pressure-holding time: 10 min. Based on the experimental objective that the higher the polysaccharide yield, the better, the optimal experimental scheme should be determined according to a level corresponding to the largest K value for each factor. K₁, K₂, and K₃ correspond to level 1, level 2, and level 3, respectively. As shown in Table 3, the factors including the inoculum size, the fermentation time, the pressure, and the pressure-holding time have significant impacts on the polysaccharide yield. Thus, the highest levels for these factors should be selected when the optimal levels are determined: inoculum size: 5%, fermentation time: 2 d, pressure: 500 MPa, and pressure-holding time: 10 min. However, the material-to-liquid ratio has no significant impact on the polysaccharide yield. Thus, to maximize the processing efficiency, a material-to-liquid ratio of 1:5 is selected.

The optimal process selected above was validated according to the experimental steps in Example 1, and a corresponding polysaccharide extraction yield was determined.

### Comparative Example 1

A hot water extraction method for a Rhizoma dioscoreae polysaccharide was provided, including the following steps:

20 g of Rhizoma dioscoreae was weighed, mixed with water according to a material-to-liquid ratio of 10 mL/g, and beaten to produce a Rhizoma dioscoreae slurry. The Rhizoma dioscoreae slurry was subjected to extraction in hot water at 80° C. for 3 h and then centrifuged at 4,000 r/min. A resulting supernatant was collected, and concentrated to a volume ⅕ of an original volume. A resulting concentrate was deproteinized by Sevag method (a Sevag reagent was added in a volume ¼ of a volume of the concentrate, and shaking was conducted vigorously for 30 min. The Sevag reagent was prepared by mixing chloroform and n-butanol at 4:1), subjected to organic reagent removal through rotary evaporation, and then subjected to dialysis in pure water for 48 h using a dialysis bag with a molecular weight cut-off of 8,000 Da. A resulting dialysate was collected and dried to produce an extract.

### Comparative Example 2

An ultra-high pressure treatment-based extraction method for a Rhizoma dioscoreae polysaccharide was provided, including the following steps: 20 g of Rhizoma dioscoreae was weighed, mixed with water according to a material-to-liquid ratio of 6 mL/g, and beaten to produce a Rhizoma dioscoreae slurry. The Rhizoma dioscoreae slurry was placed in a polyethylene bag, sealed, and subjected to an ultra-high pressure treatment under a pressure of 500 MPa with a pressure-holding time of 10 min. A specified amount of water was added according to a final material-to-liquid ratio of 10 mL/g, and extraction was conducted in hot water at 80° C. for 3 h. Centrifugation was conducted, and a resulting supernatant was collected and concentrated to a volume ⅕ of an original volume. A resulting concentrate was deproteinized by Sevag method (a Sevag reagent was added in a volume ¼ of a volume of the concentrate, and shaking was conducted vigorously for 30 min. The Sevag reagent was prepared by mixing chloroform and n-butanol at 4:1), subjected to organic reagent removal through rotary evaporation, and then subjected to dialysis in pure water for 48 h using a dialysis bag with a molecular weight cut-off of 8,000 Da. A resulting dialysate was collected and dried to produce an extract.

### Comparative Example 3

A fermentation-based extraction method for a Rhizoma dioscoreae polysaccharide was provided, including the following steps: 20 g of Rhizoma dioscoreae was weighed, mixed with water according to a material-to-liquid ratio of 6 mL/g, and beaten to produce a Rhizoma dioscoreae slurry. A Lactiplantibacillus plantarum subsp. plantarum suspension was inoculated at an inoculum size of 4% into the Rhizoma dioscoreae slurry (a Lactiplantibacillus plantarum subsp. plantarum strain, a method for preparing the Lactiplantibacillus plantarum subsp. plantarum suspension, and an inoculation method were all the same as those in Example 1), and fermentation was conducted at 37° C. for 2 d. After the fermentation was completed, water was added according to a final water/fermentation broth ratio of 10 mL/g, and extraction was conducted in hot water at 80° C. for 3 h. Centrifugation was conducted at 4,000 r/min, and a resulting supernatant was collected and concentrated to a volume ⅕ of an original volume. A resulting concentrate was deproteinized by Sevag method (a Sevag reagent was added in a volume ¼ of a volume of the concentrate, and shaking was conducted vigorously for 30 min. The Sevag reagent was prepared by mixing chloroform and n-butanol at 4:1), subjected to organic reagent removal through rotary evaporation, and then subjected to dialysis in pure water for 48 h using a dialysis bag with a molecular weight cut-off of 8,000 Da. A resulting dialysate was collected and dried to produce an extract.

The extracts obtained in Example 1 and Comparative Examples 1 to 3 were each tested for a polysaccharide yield, a lipase-inhibiting activity, and an amylase-inhibiting activity.

The polysaccharide extraction yield and the total sugar content were determined by the same processes as in Test method 1.

Test method 2: Amylase-inhibiting activity determination test: 100 μL of a Rhizoma dioscoreae polysaccharide (0.1 mg/mL to 20 mg/mL) solution and 100 μL of a α-amylase (5 U/mL) solution were mixed and incubated at 37° C. for 15 min. 100 μL of a 2 mg/mL soluble starch solution was then added, and incubation was conducted for 10 min. 225 μL of a DNS solution was added, and incubation was conducted in a boiling-water bath for 5 min to terminate a reaction. Then, a resulting mixture was cooled to room temperature, diluted 5-fold with phosphate buffered saline (PBS) (75 Mm, pH 7.4), and tested for absorbance at 540 nm by an ultraviolet-visible spectrophotometer. Acarbose was adopted as a positive control.

$$Inhibition rate = ( 1 - \frac{A 2 - A 1}{A 3 - A 4} ) * 100$$

where A1: Rhizoma dioscorede polysaccharide+α-amylase+starch, A2: Rhizoma dioscoreae polysaccharide+soluble starch, A3: soluble starch+α-amylase, and A4: soluble starch.

Test method 3: Pancreatic lipase-inhibiting activity determination test: 120 μL of a soluble dietary fiber (0.1 mg/mL to 20 mg/mL) solution and 40 μL of a pancreatic lipase solution (1 mg/mL) were mixed and incubated at 37° C. for 15 min. Then 40 μL of a 2 mM 4-nitrophenyl butyrate (PNPB) solution was added, and incubation was conducted at 37° C. for 15 min. The absorbance at 405 nm was determined. Orlistat was adopted as a positive control.

$$Inhibition rate = ( 1 - \frac{A 2 - A 1}{A 3 - A 4} ) * 100$$

where A1: Rhizoma dioscoreae polysaccharide+pancreatic lipase+PNPB, A2: Rhizoma dioscoreae polysaccharide+PNPB, A3: pancreatic lipase+PNPB, and A4: PNPB.

TABLE 4
Influence of different extraction processes on a yield and
an activity of the Rhizoma Dioscoreae polysaccharide

| | | | IC₅₀ value for a | |
|---|---|---|---|---|
| | | Total | pancreatic | IC₅₀ value for an |
| | Polysaccharide | sugar | lipase-inhibiting | amylase-inhibiting |
| Sample | extraction yield | content | activity | activity |
| Example 1 | 10.26% | 80.18% | 14.13 mg/mL | 7.47 mg/mL |
| Comparative | 3.66% | 68.33% | 22.54 mg/mL | 20.20 mg/mL |
| Example 1 | | | | |
| Comparative | 5.42% | 77.07% | 21.31 mg/mL | 11.32 mg/mL |
| Example 2 | | | | |
| Comparative | 6.65% | 85.53% | 16.64 mg/mL | 9.80 mg/mL |
| Example 3 | | | | |

As shown in Table 4, compared with Comparative Examples 1 to 3, the preparation method of the present disclosure enables a Rhizoma dioscoreae polysaccharide extraction yield of 10% or more, which is significantly higher than the Rhizoma dioscoreae polysaccharide extraction yield of 3.66% to 6.65% in the prior art. Moreover, the Rhizoma dioscoreae polysaccharide produced by the method of the present disclosure exhibits a significantly-enhanced inhibitory activity against pancreatic lipase and amylase. It can be seen that the Rhizoma dioscoreae polysaccharide extracted by the method of the present disclosure demonstrates a significantly-improved extraction yield and activity.

The above are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. A method for preparing of a highly-active Rhizoma dioscoreae polysaccharide, comprising the following steps:
subjecting a Rhizoma dioscoreae slurry to ultra-high pressure treatment to produce an ultra-high pressure-treated Rhizoma dioscoreae slurry;
inoculating Lactiplantibacillus plantarum subsp. plantarum into the ultra-high pressure-treated Rhizoma dioscoreae slurry, and conducting fermentation to produce a Rhizoma dioscoreae fermentation broth; and
subjecting the Rhizoma dioscoreae fermentation broth to hot water extraction and then centrifugation to obtain a supernatant, concentrating the supernatant to obtain a concentrated solution, and drying the concentrated solution to produce the Rhizoma dioscoreae polysaccharide.

2. The method according to claim 1, wherein the method further comprises mixing fresh Rhizoma dioscoreae and water at a mass ratio of 1:0.5-3 to obtain a mixture, and pulping the mixture to produce an preliminary Rhizoma dioscoreae slurry.

3. The method according to claim 1, wherein the method further comprises mixing the preliminary Rhizoma dioscoreae slurry and water to produce the Rhizoma dioscoreae slurry before the ultra-high pressure treatment; and in the Rhizoma dioscoreae slurry, a mass-to-volume ratio of the fresh Rhizoma dioscoreae to total water is 1:4 to 1:8.

4. The method according to claim 1, wherein the ultra-high pressure treatment is conducted under a pressure of 200 MPa to 600 MPa with a pressure-holding time of 5 min to 11 minutes.

5. The method according to claim 1, wherein the Lactiplantibacillus plantarum subsp. plantarum is cultured in an MRS broth medium to produce a bacterial suspension for use.

6. The method according to claim 1, wherein concentration of the Lactiplantibacillus plantarum subsp. plantarum is 0.5×10⁹ cfu/mL to 1.5×10⁹ cfu/mL.

7. The method according to claim 1, wherein a inoculation percentage of a mass of fresh Rhizoma dioscoreae to a volume of a suspension of the Lactiplantibacillus plantarum subsp. plantarum is 2% to 6%.

8. The method according to claim 1, wherein the fermentation is conducted at 35° C. to 40° C.

9. The method according to claim 1, wherein the fermentation is conducted for 1 day to 4 days.

10. The method according to claim 1, wherein before the Lactiplantibacillus plantarum subsp. plantarum is inoculated for the fermentation, the ultra-high pressure-treated Rhizoma dioscoreae slurry is pasteurized at 70° C. to 90° C. for 20 minutes to 40 minutes.

11. The method according to claim 1, wherein water is added at a final amount of 8 mL to 15 mL per 1 g of fresh Rhizoma dioscoreae to the Rhizoma dioscoreae fermentation broth to conduct the hot water extraction.

12. The method according to claim 1, wherein the hot water extraction is conducted at 80° C. to 90° C.

13. The method according to claim 1, wherein the hot water extraction is conducted for 2 hours to 5 hours.

14. The method according to claim 1, wherein the concentrating is conducted to a volume ⅙ to ⅓ of an original volume.

15. The method according to claim 1, wherein the method further comprises deproteinizing the concentrated solution by Sevag method, subjecting to organic reagent removal through rotary evaporation, and then subjecting to dialysis in water using a dialysis bag with a molecular weight cut-off of 7,000 Da to 9,000 Da for 36 hours to 60 hours to obtain a dialysate, drying the dialysate to produce the Rhizoma dioscoreae polysaccharide.
